(19) United States
(12) Patent Application Publication
(10) Pub. No.: US 2023/0120480 A1
(43) Pub. Date: Apr. 20, 2023

(54) METHODS FOR REMOVING CONTAMINANTS FROM PLANT-DERIVED PHARMACEUTICALS

(71) Applicant: SORBENT TECHNOLOGIES, INC., Norcross, GA (US)

(72) Inventor: Robert R. Kerr, Duluth, GA (US)

(21) Appl. No.: 17/960,379

(22) Filed: Oct. 5, 2022

Related U.S. Application Data
(60) Provisional application No. 63/253,099, filed on Oct. 6, 2021.

(51) Int. Cl. A61K 36/185

(52) U.S. Cl. CPC A61K 36/185; A61K 2236/50; A61K 2236/35

(57) ABSTRACT

Methods of removing contaminants such as pesticides, herbicides, and fungicides from plant-derived pharmaceuticals, such as cannabis-derived pharmaceuticals, are disclosed. Plant-derived pharmaceuticals, such as cannabis-derived pharmaceuticals, and methods of using the plant-derived pharmaceuticals are also disclosed.

### CROSS-REFERENCE TO RELATED APPLICATIONS

This patent application claims the benefit of priority to U.S. Provisional Patent Application Ser. No. 63/253,099 filed on Oct. 6, 2021, and entitled “METHODS FOR REMOVING CONTAMINANTS FROM PLANT-DERIVED PHARMACEUTICALS,” the subject matter of which is hereby incorporated by reference in its entirety.

### FIELD OF THE INVENTION

The present invention relates generally to methods of removing contaminants such as pesticides, herbicides, and fungicides from plant-derived pharmaceuticals such as cannabis-derived pharmaceuticals. The present invention further relates to plant-derived pharmaceuticals and methods of using the plant-derived pharmaceuticals.

### BACKGROUND OF THE INVENTION

The plant genus Cannabis is cultivated for a variety of reasons, one of which is to obtain the organic substances made by the plant called Cannabinoids that are unique to the genus.

Pesticides, synergists, herbicides, and fungicides collectively referred to herein as “contaminants,” are used by cultivators to increase crop yield and crop quality. Some of the contaminants are applied directly to the plants. Some contaminants pollute the crop after application to crops in adjacent fields. Some contaminants are persistent and remain in the soil from previous crops.

Cannabinoids are extracted from the plant matrix by a variety of methods, the most prominent of which are: solvent extraction and supercritical fluid extraction. In any case, the cannabinoids are extracted and concentrated from the plant matrix with a resulting oily, semi-solid material.

The extraction processes for removing and concentrating cannabinoids also remove and concentrate the toxic contaminants. All solvents extract a relatively broad range of substances. Further, substances with similar characteristics are always extracted together. In terms of solubility in common solvents, the cannabinoids and the contaminants have very similar properties, and consequently, are typically extracted together.

Health authorities in some states have set permissible limits on the concentration of individual contaminants in Cannabis. In the Oregon Health Authorities Technical Report 8964, some 59 contaminants, primarily pesticides, synergists, and fungicides, must be tested in Cannabis and Cannabis-containing products before they can be sold.

Testing for contaminants in Cannabis products has commenced in several states in the United States. It has been found that the extracts contain relatively elevated amounts of contaminants that are a danger to human health.

In general, the methods available to obtain Cannabis extracts with safe levels of contaminants are: (1) pesticide-free crop growth, (2) selective extraction, (3) selective adsorption, and (4) chromatographic purification. It is also possible to destroy contaminants by selective chemical reaction; however, this method is not typically employed since there are very few situations in which the desired components differ significantly in their chemical reactivity towards specific reactant relative to the contaminants.

Generally speaking, dried material of Cannabis Saliva contains about 10% cannabinoids and Cannabis Indica (or hemp) contains 3-5% cannabinoids. Even when the raw plant materials are measured to have low or acceptable levels of contaminants, after known extraction and concentration techniques, the Cannabis extracts show a significant increase in contaminant concentration using the conventional techniques discussed above. Generally, the contaminant concentration is typically increased by a factor of 10-fold using the conventional techniques discussed above.

The initial solvent extractions of Cannabis and Hemp, particularly ethanolic extracts, are usually a muddy dark-green, dark-brown color. Before solvent removal, the extract is filtered to remove undesirable extracted substances using multiple stages of filtration. Dewaxing the raw extracts by temperature reduction to −40° C. to −60° C., followed by depth filters at that temperature, is frequently applied first. Then, to remove chlorophyll, a filter with activated carbon and/or activated alumina is used with the former being generally acknowledged to be the most effective. To remove particulates and debris both from the initial extract, as well as from the previous filter aids, a second and third stage of filters consisting of diatomaceous earth and depth filters is used. These filtration steps do not remove any of the regulated contaminants.

U.S. Pat. No. 11,026,985, entitled “Methods for Removing Contaminants from Plant-Derived Pharmaceuticals” discloses methods of removing contaminants (e.g., pesticides, herbicides, fungicides, and synergists). Unexpectedly, it has recently been discovered that when using the methods described in U.S. Pat. No. 11,026,985 on unfiltered alcoholic Cannabis extracts (or distillates) from California USA, the resulting extracts darken slightly to a deep red color. It should be noted that when using the methods described in U.S. Pat. No. 11,026,985 on filtered alcoholic Hemp extracts and distillates, as well as commercially obtained extracts of hops, this darkening to a deep red color does not occur. Further, this extract darkening has not been observed to date with hydrocarbon extracts (filtered or unfiltered) or with carbon dioxide supercritical fluid extraction (SFE) extracts (filtered or unfiltered) of either Cannabis or Hemp plant material. This extract darkening is undesirable commercially in the highly competitive Cannabis extract market where clarity and color are highly desirable.

There is a need for simple, effective, sate, and environmentally friendly methods of removing contaminants from plant-derived pharmaceuticals such as cannabis-derived extracts, so as to result in extracts having a desired color (e.g., without the extract darkening to a deep red color) even when the plant-derived pharmaceutical is derived from unfiltered alcoholic Cannabis extracts (or distillates) from California USA.

### SUMMARY OF THE INVENTION

The present invention is directed to simple, effective, safe, and environmentally friendly methods of removing contaminants from plant-derived pharmaceuticals. The disclosed methods efficiently and safely enable proper removal of contaminants such as pesticides, herbicides, fungicides, etc. from plant-derived pharmaceuticals. The disclosed methods also provide a cost-effective and convenient method of removing contaminants from plant-derived pharmaceuticals.

In some embodiments of the present invention, the method of removing contaminants from plant-derived pharmaceuticals comprises: reacting one or more contaminants within a plant-derived extract with a hydrolytic agent (e.g., ammonium hydroxide) in a polar solvent (e.g., an alcohol) to form one or more contaminant by-products; and separating the one or more contaminant by-products from the plant-derived extract so as to obtain a contaminant-reduced, plant-derived extract, wherein the contaminant-reduced, plant-derived extract does not have a darkening red color.

The present invention is further directed to contaminant-reduced, plant-derived extracts formed using the herein-disclosed methods of removing contaminants from plant-derived pharmaceuticals wherein the contaminant-reduced, plant-derived extract does not have a darkening red color. In some embodiments of the present invention, the contaminant-reduced, plant-derived extracts formed using the herein-disclosed methods of removing contaminants from plant-derived pharmaceuticals comprise cannabinoids.

The present invention is even further directed to methods of using contaminant-reduced, plant-derived extracts to treat a patient suffering from one or more diseases or disorders wherein the contaminant-reduced, plant-derived extract does not have a darkening red color. In some embodiments of the present invention, the method of using contaminant-reduced, plant-derived extracts comprises administering an effective amount of the contaminant-reduced, plant-derived extract to the patient, and the contaminant-reduced, plant-derived extract (i) comprises one or more cannabinoids, and (ii) does not have a darkening red color.

These and other features and advantages of the present invention will become apparent after a review of the following detailed description of the disclosed embodiments and the appended claims.

### BRIEF DESCRIPTION OF THE FIGURES

The present invention is further described with reference to the appended figure, wherein:

FIGS. 1A-1B depict an exemplary process flowchart showing steps for the removal of contaminants from plant-derived pharmaceuticals using ammonium hydroxide as the reaction hydrolytic agent;

FIG. 2 depicts another exemplary process flowchart showing steps for the removal of contaminants from plant-derived pharmaceuticals using ammonium hydroxide as the reaction hydrolytic agent; and

FIG. 3 depicts another exemplary process flowchart showing steps for the removal of contaminants from plant-derived pharmaceuticals using potassium hydroxide and ammonium hydroxide as the reaction hydrolytic agents.

### DETAILED DESCRIPTION OF THE INVENTION

The present invention is directed towards methods of removing contaminants from plant-derived pharmaceuticals. The present invention is further directed towards plant-derived pharmaceuticals obtained using the herein-described methods, and methods of using the plant-derived pharmaceuticals.

### I. Methods of Removing Contaminants From Plant-Derived Pharmaceuticals

The present invention is directed towards methods of removing contaminants from plant-derived pharmaceuticals.

The functional groups of the cannabinoids are: (1) alkanes, (2) alkenes, (3) aromatic rings, (4) phenolic hydroxyls, (5) carboxylic acids, and (6) aryl alkyl ethers. Neutral cannabinoids (i.e., without a carboxylic acid functional group) are insoluble in water.

Without special conditions, the functional groups of the cannabinoids are not subject to attack by most nucleophiles.

The hydroxides used in the present methods include, but are not limited to, hydroxides such as ammonium hydroxide, and tetraalkylammonium hydroxide salts such as tetramethylammonium hydroxide, tetraethylammonium hydroxide, tetrapropylammonium hydroxide, etc. All of these substances are: (1) relatively inexpensive, (2) readily soluble in water and the lower molecular weight alcohols (methanol, ethanol, isopropyl alcohol, and n-propanol), and (3) strong nucleophiles and agents of hydrolysis.

Pesticides contain a wide range of functional groups. Most pesticides contain at least one functional group that can be attacked by nucleophiles such as an alkali metal hydroxide or ammonium hydroxide in alcoholic solution. Of the 59 pesticides, fungicides, and synergists with levels in Cannabis regulated by the State of Oregon, 45 are subject to nucleophilic attack under these conditions, 6 might be, and 8 are not. Once a functional group has been transformed by such a reaction, the biological activity is usually lost.

Additionally, ammonium hydroxide forms a Schiff Base with all substances that contain a carbonyl function including aldehydes, ketones, carboxylic acids, esters, anhydrides, acyl halides, amides, and quinones. Schiff Base formation is reversible by acids. Further, these Schiff Bases are not soluble in alkanes.

Several of the non-hydrolysable contaminants possess carbonyl groups that react with ammonium hydroxide to form water soluble substances that are not extractable by alkanes.

Many of the non-Cannabinoid components in the extract react with ammonium hydroxide to form water soluble substances that are not extractable by alkanes.

None of the Cannabinoids of interest contain any functional groups that can be attacked by strong nucleophiles such as an ammonium hydroxide in alcoholic solution. None of the Cannabinoids of interest possess a carbonyl group that will react with ammonium hydroxide.

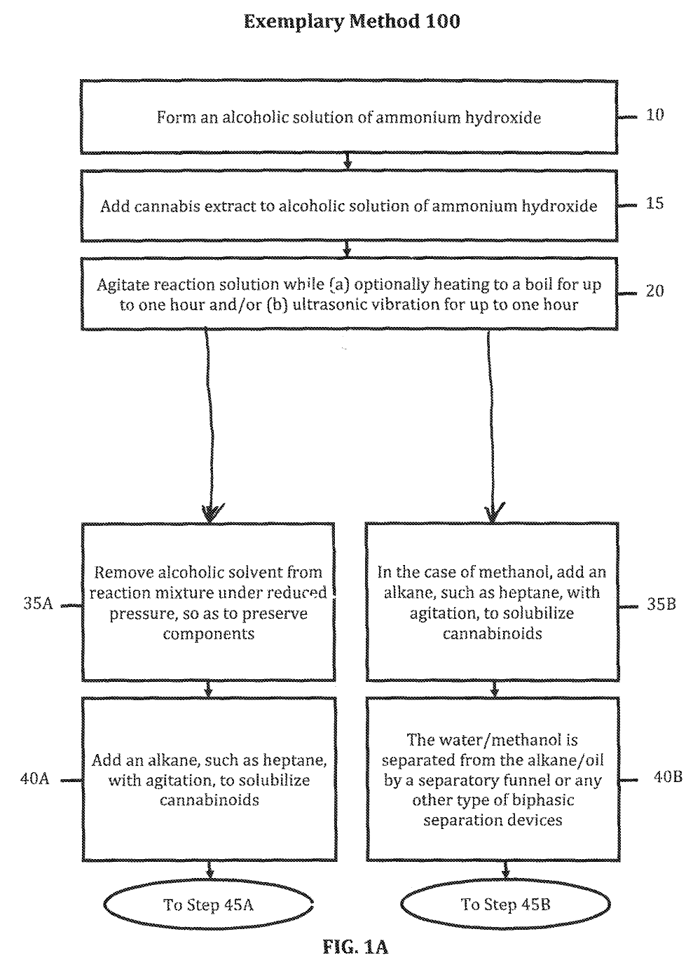

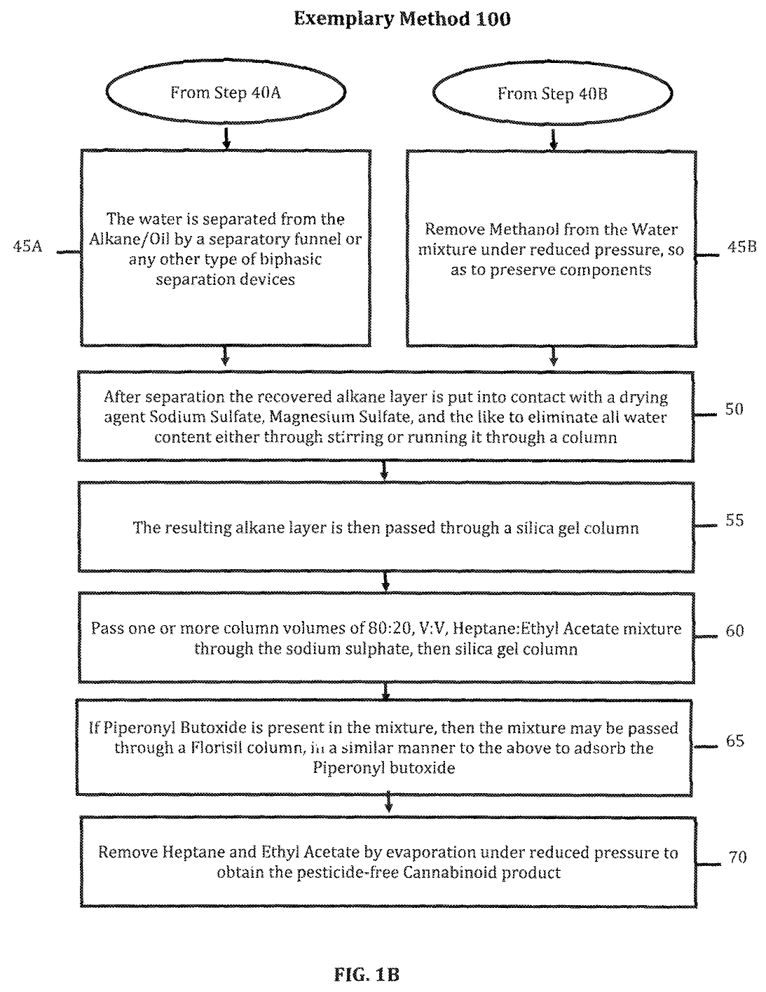

The present invention provides what is believed to be the best solution to eliminate all hydrolyzable pesticides, which is nearly all pesticides, from Cannabis extracts, as well as other undesirable components of the original extract, which later may yield a colored substance. The method of entirely removing all hydrolyzable pesticides from a given cannabis extract comprises a reaction step, wherein one or more contaminants are reacted with a hydroxide solution, such as ammonium hydroxide, followed by an adsorbent based clean-up step to eliminate any pesticide fragments and any remaining pesticides. This desired method is described below, as well as shown in FIGS. 1A-1B,

As shown in FIG. 1A, exemplary method 100 of the present invention starts at step 10, wherein an alcoholic solution of a hydroxide, such as ammonium hydroxide, is formed, From step 10, exemplary method 100 proceeds to step 15, wherein a given Cannabis extract is added to the alcoholic solution of ammonium hydroxide. From step 15, exemplary method 100 proceeds to step 20, wherein the resulting reaction solution is mechanically agitated optionally with ultrasonic vibration with sufficient molar excess of ammonium hydroxide for a sufficient time to insure complete reaction, typically for up to an hour or more.

If the alcoholic solvent used is methanol, exemplary method 100 can proceed to step 35B, where an alkane solvent (such as pentane, hexane, heptane, and the like) is added directly to the reaction mixture since both methanol and water are immiscible with an alkane and the substances of interest are very soluble in the alkane.

From step 35B, exemplary method 100 proceeds to step 40B, wherein the water/alcohol is separated from the alkane/oil by a separatory funnel or any other type of biphasic separation devices. From step 40B, exemplary method 100 proceeds to step 45B shown in FIG. 1B, wherein the methanol is removed from the water/methanol mixture by evaporation under reduced pressure for possible reuse in future contaminant-removal cycles (i.e., in step 10). Other alcohols, such as ethanol, isopropanol, n-propanol and so on, are miscible with alkanes so this only works with methanol.

From step 45A or 45B, exemplary method 100 proceeds to step 50, wherein, the recovered alkane/oil layer is put into contact with a drying agent, such as sodium sulfate, magnesium sulfate, and the like, to eliminate all water content. In this step, the powdered drying agent can be added to the recovered alkane/oil layer, agitated, and then filtered off. Alternatively, the drying agent can be added to a chromatographic column and the recovered alkane/oil layer pumped through the filled column.

From step 50, exemplary method 100 proceeds to step 55, wherein the dried alkane/oil layer is passed through a silica gel column. From step 55, exemplary method 100 proceeds to step 60, wherein one or more, typically several, column volumes of a mixture of heptane and ethyl acetate (80:20 v/v) is passed through the silica gel column. The silica gel column (i) allows the non-polar Cannabinoids to pass through unretained, (ii) absorbs any water, (iii) retains fragments of the hydrolyzed pesticides, and (iv) retains unhydrolyzable polar pesticides.

If piperonyl butoxide, a non-toxic(i.e., to humans) ingredient commonly added to commercially available insecticides, is believed to be present, exemplary method 100 proceeds from step 60 to step 65, wherein the resulting mixture is passed through a FLORISIL® (magnesium silicate) column to absorb the piperonyl butoxide.

It should be noted that the vast majority of commercially available pesticides contain piperonyl butoxide because it increases the toxicity of the pesticides to insects. Piperonyl butoxide is not affected by the above-described hydrolysis step and it elutes from the silica gel column at the same time as the Cannabinoids. Piperonyl butoxide has a polyether like side chain. FLORISIL,® is a tradename for micron sized heat activated (675° C.) magnesium silicate.

From step 65, exemplary method 100 proceeds to step 70, wherein the heptane and ethyl acetate are removed by evaporation under reduced pressure to obtain a pesticide-free Cannabinoid product. If it is known that no piperonyl butoxide is present in a given sample, exemplary method 100 may proceed directly to step 70 (i.e., without step 65 shown in FIG. 18).

The above-described exemplary method 100 has been found to effectively remove many contaminants including, but not limited to, many of the pesticides listed in Table 1 below.

TABLE 1
Pesticides in Cannabis Regulated by the State of Oregon

| | | Chemical | | | |
|---|---|---|---|---|---|
| | | Abstract | | | |
| | Pesticide | Services | Action | | Group Subject |
| | Common | (CAS) | Level | | To Nucleophilic |
| No. | Name | Registry | (ppm) | Chemical Structure | Attack Present? |
| 1 | Abamectin | 71751-41-2 | 0.5 | 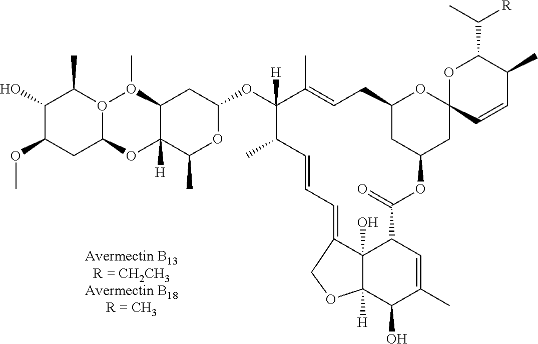 | Yes |
| 2 | Acephate | 30560-19-1 | 0.4 | 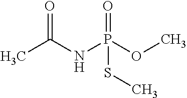 | Yes |
| 3 | Acequinocyl | 57960-19-7 | 2 | 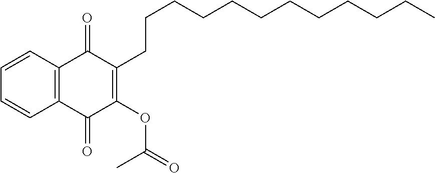 [SMILES: CCCCCCCCCCCCC1=C(OC(C)=O)C(=O)c2ccccc2C1=O] | Yes |
| 4 | Acetamiprid | 135410-20-7 | 0.2 | 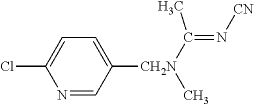 | Yes |
| 5 | Aldicarb | 116-06-3 | 0.4 | 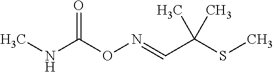 | Yes |
| 6 | Azoxystrobin | 131860-33-8 | 0.2 | 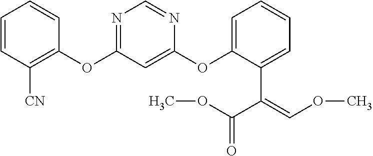 [SMILES: CO/C=C(/C(=O)OC)c1ccccc1Oc1cc(Oc2ccccc2C#N)ncn1] | Yes |
| 7 | Bifenazate | 149877-41-8 | 0.2 | 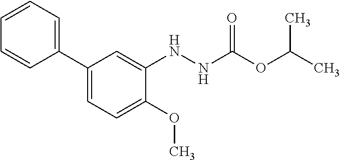 [SMILES: COc1ccc(-c2ccccc2)cc1NNC(=O)OC(C)C] | Yes |
| 8 | Bifenthrin | 82657-04-3 | 0.2 | 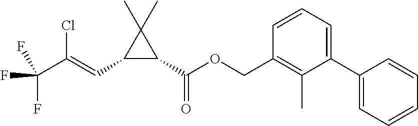 | Yes |
| 9 | Boscalid | 188425-85-6 | 0.4 | 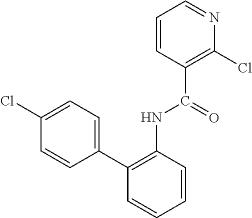 [SMILES: O=C(Nc1ccccc1-c1ccc(Cl)cc1)c1cccnc1Cl] | No |
| 10 | Carbaryl | 63-25-2 | 0.2 | 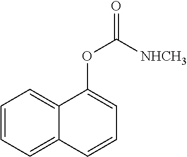 [SMILES: CNC(=O)Oc1cccc2ccccc12] | Yes |
| 11 | Carbofuran | 1563-66-2 | 0.2 | 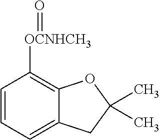 | Yes |
| 12 | Chlorantranili- prole | 500008-45-7 | 0.2 | 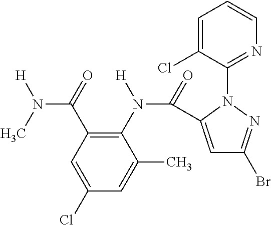 [SMILES: CNC(=O)c1cc(Cl)cc(C)c1NC(=O)c1cc(Br)nn1-c1ncccc1Cl] | Yes |
| 13 | Chlorfenapyr | 122453-73-0 | 1 | 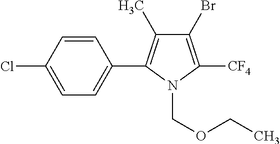 | No |
| 14 | Chlorpyrifos | 2921-88-2 | 0.2 | 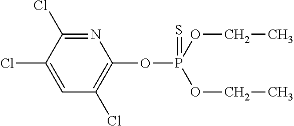 [SMILES: CCOP(=S)(OCC)Oc1nc(Cl)c(Cl)cc1Cl] | Yes |
| 15 | Clofentezine | 74115-24-5 | 0.2 | 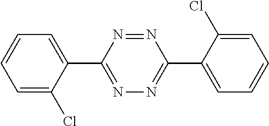 [SMILES: Clc1ccccc1-c1nnc(-c2ccccc2Cl)nn1] | May-be |
| 16 | Cyfluthrin | 68359-37-5 | 1 | 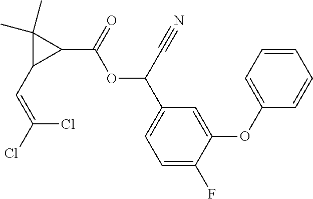 [SMILES: CC1(C)C(C=C(Cl)Cl)C1C(=O)OC(C#N)c1ccc(F)c(Oc2ccccc2)c1] | Yes |
| 17 | Cypermethrin | 52315-07-8 | 1 | 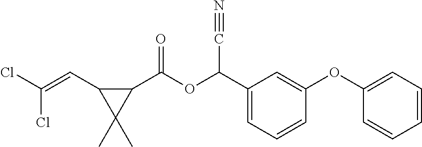 [SMILES: CC1(C)C(C=C(Cl)Cl)C1C(=O)OC(C#N)c1cccc(Oc2ccccc2)c1] | Yes |
| 18 | Daminozide | 1596-84-5 | 1 | 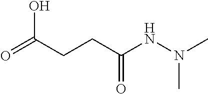 | Yes |
| 19 | DDVP (Dichlorvos) | 62-73-7 | 0.1 | 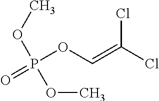 | Yes |
| 20 | Diazinon | 333-41-5 | 0.2 | 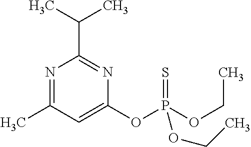 [SMILES: CCOP(=S)(OCC)Oc1cc(C)nc(C(C)C)n1] | Yes |
| 21 | Dimethoate | 60-51-5 | 0.2 | 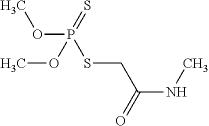 | Yes |
| 22 | Ethoprophos | 13194-48-4 | 0.2 | 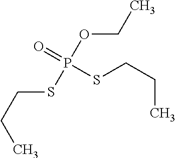 | Yes |
| 23 | Etofenprox | 80844-07-1 | 0.4 | 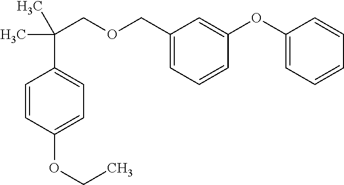 [SMILES: CCOc1ccc(C(C)(C)COCc2cccc(Oc3ccccc3)c2)cc1] | No |
| 24 | Etoxazole | 153233-91-1 | 0.2 | 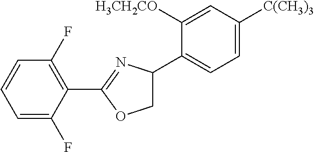 | No |
| 25 | Fenoxycarb | 72490-01-8 | 0.2 | 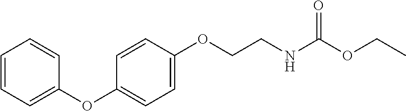 [SMILES: CCOC(=O)NCCOc1ccc(Oc2ccccc2)cc1] | Yes |
| 26 | Fenpyroximate | 134098-61-6 | 0.4 | 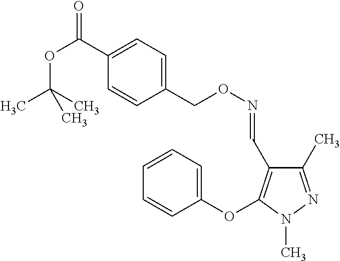 [SMILES: Cc1nn(C)c(Oc2ccccc2)c1/C=N/OCc1ccc(C(=O)OC(C)(C)C)cc1] | Yes |
| 27 | Fipronil | 120068-37-3 | 0.4 | 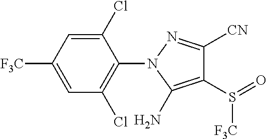 [SMILES: N#Cc1nn(-c2c(Cl)cc(C(F)(F)F)cc2Cl)c(N)c1S(=O)C(F)(F)F] | Yes |
| 28 | Flonicamid | 158062-67-0 | 1 | 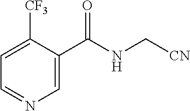 [SMILES: N#CCNC(=O)c1cnccc1C(F)(F)F] | May-be |
| 29 | Fludioxonil | 131341-86-1 | 0.4 | 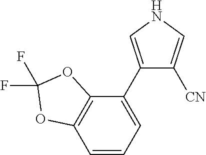 [SMILES: N#Cc1c[nH]cc1-c1cccc2c1OC(F)(F)O2] | No |
| 30 | Hexythiazox | 78587-05-0 | 1 | 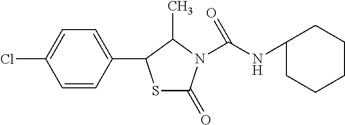 [SMILES: CC1C(c2ccc(Cl)cc2)SC(=O)N1C(=O)NC1CCCCC1] | Yes |
| 31 | Imazalil | 35554-44-0 | 0.2 | 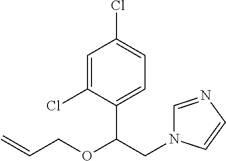 [SMILES: C=CCOC(Cn1ccnc1)c1ccc(Cl)cc1Cl] | May-be |
| 32 | Imidacloprid | 138261-41-3 | 0.4 | 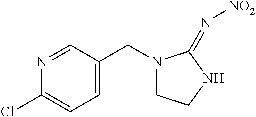 [SMILES: O=[N+]([O-])/N=C1\NCCN1Cc1ccc(Cl)nc1] | Yes |
| 33 | Kresoxim- methyl | 143390-89-0 | 0.4 | 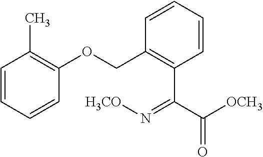 [SMILES: CO/N=C(/C(=O)OC)c1ccccc1COc1ccccc1C] | Yes |
| 34 | Malathion | 121-75-5 | 0.2 | 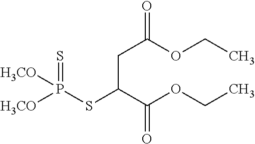 [SMILES: CCOC(=O)CC(SP(=S)(OC)OC)C(=O)OCC] | Yes |
| 35 | Metalaxyl | 57837-19-1 | 0.2 | 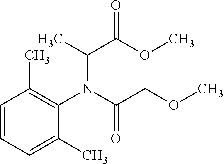 [SMILES: COCC(=O)N(c1c(C)cccc1C)C(C)C(=O)OC] | Yes |
| 36 | Methiocarb | 2032-65-7 | 0.2 | 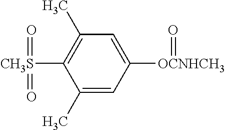 | Yes |
| 37 | Methomyl | 16752-77-5 | 0.4 | 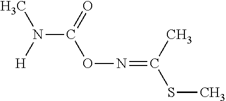 | Yes |
| 38 | Methyl parathion | 298-00-0 | 0.2 | 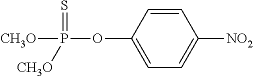 | Yes |
| 39 | MGK-264 | 113-48-4 | 0.2 | 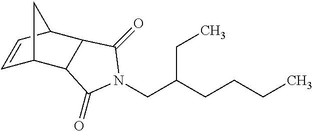 [SMILES: CCCCC(CC)CN1C(=O)C2C3C=CC(C3)C2C1=O] | May-be |
| 40 | Myclobutanil | 88671-89-0 | 0.2 | 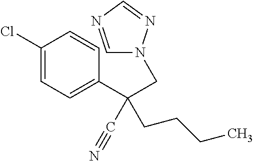 [SMILES: CCCCC(C#N)(Cn1cncn1)c1ccc(Cl)cc1] | Yes |
| 41 | Naled | 300-76-5 | 0.5 | 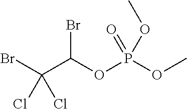 | Yes |
| 42 | Oxamyl | 23135-22-0 | 1 | 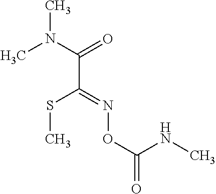 | Yes |
| 43 | Paclobutrazol | 76738-62-0 | 0.4 | 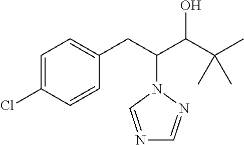 [SMILES: CC(C)(C)C(O)C(Cc1ccc(Cl)cc1)n1cncn1] | May-be |
| 44 | Permethrins | 52645-53-1 | 0.2 | 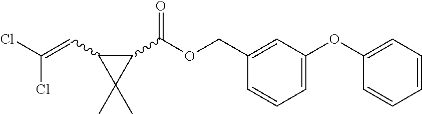 [SMILES: CC1(C)C(C=C(Cl)Cl)C1C(=O)OCc1cccc(Oc2ccccc2)c1] | Yes |
| 45 | Phosmet | 732-11-6 | 0.2 | 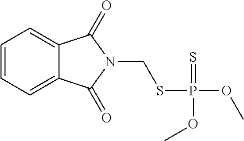 [SMILES: COP(=S)(OC)SCN1C(=O)c2ccccc2C1=O] | Yes |
| 46 | Piperonyl butoxide | 51-03-6 | 2 | 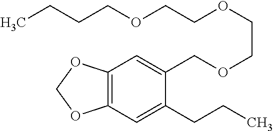 [SMILES: CCCCOCCOCCOCc1cc2c(cc1CCC)OCO2] | No |
| 47 | Prallethrin | 23031-36-9 | 0.2 | 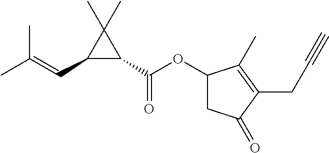 [SMILES: C#CCC1=C(C)C(OC(=O)[C@@H]2[C@@H](C=C(C)C)C2(C)C)CC1=O] | Yes |
| 48 | Propiconazole | 60207-90-1 | 0.4 | 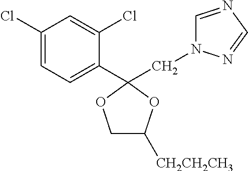 | No |
| 49 | Propoxur | 114-26-1 | 0.2 | 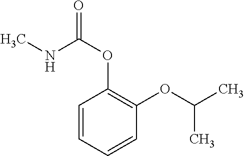 | Yes |
| 50 | Pyrethrins | 8003-34-7 | 1 | 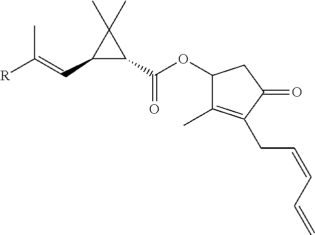 | Yes |
| 51 | Pyridaben | 96489-71-3 | 0.2 | 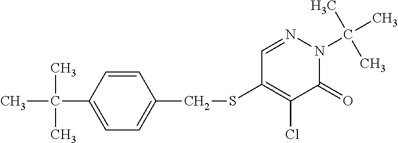 [SMILES: CC(C)(C)c1ccc(CSc2cnn(C(C)(C)C)c(=O)c2Cl)cc1] | Yes |
| 52 | Spinosad | 168316-95-8 | 0.2 | 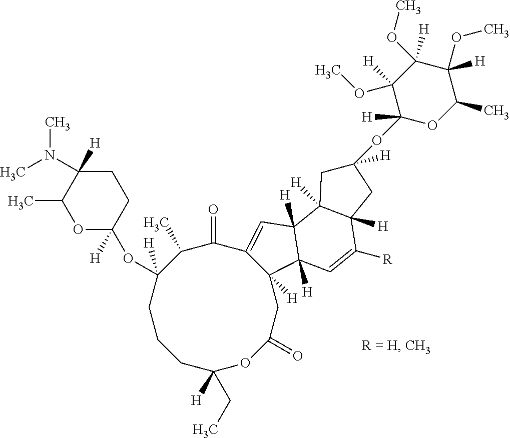 | Yes |
| 53 | Spiromesifen | 283594-90-1 | 0.2 | 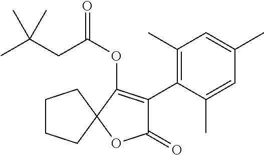 [SMILES: Cc1cc(C)c(C2=C(OC(=O)CC(C)(C)C)C3(CCCC3)OC2=O)c(C)c1] | Yes |
| 54 | Spirotetramat | 203313-25-1 | 0.2 | 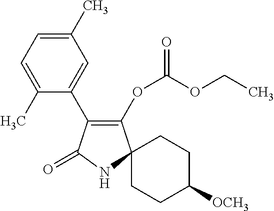 [SMILES: CCOC(=O)OC1=C(c2cc(C)ccc2C)C(=O)N[C@]12CC[C@@H](OC)CC2] | Yes |
| 55 | Spiroxamine | 118134-30-8 | 0.4 | 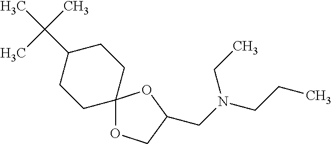 [SMILES: CCCN(CC)CC1COC2(CCC(C(C)(C)C)CC2)O1] | No |
| 56 | Tebuconazole | 80443-41-0 | 0.4 | 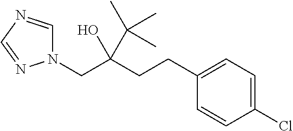 [SMILES: CC(C)(C)C(O)(CCc1ccc(Cl)cc1)Cn1cncn1] | May-be |
| 57 | Thiacloprid | 111988-49-9 | 0.2 | 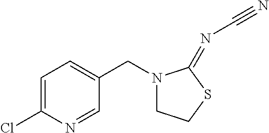 [SMILES: N#C/N=C1\SCCN1Cc1ccc(Cl)nc1] | Yes |
| 58 | Thiamethoxam | 153719-23-4 | 0.2 | 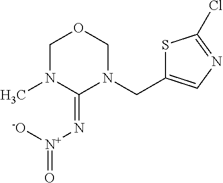 | Yes |
| 59 | Trifloxystrobin | 141517-21-7 | 0.2 | 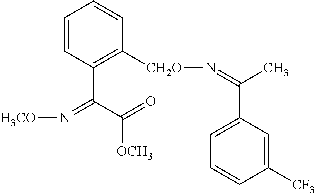 | Yes |

Further, the above-described exemplary method 100 has been found to effectively produce essentially pesticide-free cannabinoids including, but not limited to, many of the cannabinoids listed in Tables 2 and 3 below

TABLE 2
Cannabinoids

| | | Common | Water |
|---|---|---|---|
| Class | Substance | Abbreviation | Soluble? |
| Cannabichromene | Cannabichromene | CBC | No |
| Cannabichromene | Cannabichromenic acid | CBCA | Yes |
| Cannabichromene | Cannabichromevarin | CBCV | No |
| Cannabichromene | Cannabichromevarinic acid | CBCVA | No |
| Cannabichromene | Cannabicyclols | | No |
| Cannabichromene | Cannabicyclol | CBL | No |
| Cannabichromene | Cannabicyclolic acid | CBLA | Yes |
| Cannabichromene | Cannabicyclovarin | CBLV | No |
| Cannabidiols | Cannabidiol | CBD | No |
| Cannabidiols | Cannabidiol monomethylether | CBDM | No |
| Cannabidiols | Cannabidiolic acid | CBDA | No |
| Cannabidiols | Cannabidiorcol | CBD-C1 | No |
| Cannabidiols | Cannabidivarin | CBDV | No |
| Cannabidiols | Cannabidivarinic acid | CBDVA | Yes |
| Cannabielsoins | Cannabielsoic acid B | CBEA-B | No |
| Cannabielsoins | Cannabielsoin | CBE | No |
| Cannabielsoins | Cannabielsoin acid A | CBEA-A | No |
| Cannabigerols | Cannabigerol | CBG | No |
| Cannabigerols | Cannabigerol monomethylether | CBGM | No |
| Cannabigerols | Cannabigerolic acid | CBGA | Yes |
| Cannabigerols | Cannabigerolic acid | CBGAM | Yes |
| | monomethylether | | |
| Cannabigerols | Cannabigerovarin | CBGV | No |
| Cannabigerols | Cannabigerovarinic acid | CBGVA | Yes |
| Cannabinols and | Cannabinodiol | CBND | No |
| cannabinodiols | | | |
| Cannabinols and | Cannabinodivarin | CBVD | No |
| cannabinodiols | | | |
| Cannabinols and | Cannabinol | CBN | No |
| cannabinodiols | | | |
| Cannabinols and | Cannabinol methylether | CBNM | No |
| cannabinodiols | | | |
| Cannabinols and | Cannabinol-C2 | CBN-C2 | No |
| cannabinodiols | | | |
| Cannabinols and | Cannabinol-C4 | CBN-C4 | No |
| cannabinodiols | | | |
| Cannabinols and | Cannabinolic acid | CBNA | Yes |
| cannabinodiols | | | |
| Cannabinols and | Cannabiorcool | CBN-C1 | No |
| cannabinodiols | | | |
| Cannabinols and | Cannabivarin | CBV | No |
| cannabinodiols | | | |
| Cannabitriols | 10-Ethoxy-9-hydroxy- | | No |
| | delta-6a-tetrahydrocannabinol | | |
| Cannabitriols | 8,9-Dihydroxy-delta-6a- | | No |
| | tetrahydrocannabinol | | |
| Cannabitriols | Cannabitriol | CBT | No |
| Cannabitriols | Cannabitriolvarin | CBTV | No |
| Delta-8- | Delta-8- | Δ8-THC | No |
| tetrahydrocannabinols | tetrahydrocannabinol | | |
| Delta-8- | Delta-8- | Δ8-THCA | Yes |
| tetrahydrocannabinols | tetrahydrocannabinolic acid | | |
| Delta-9- | Delta-9- | THC | No |
| tetrahydrocannabinols | tetrahydrocannabinol | | |
| Delta-9- | Delta-9- | THC-C4 | No |
| tetrahydrocannabinols | tetrahydrocannabinol-C4 | | |
| Delta-9- | Delta-9- | THCA-A | Yes |
| tetrahydrocannabinols | tetrahydrocannabinolic acid A | | |
| Delta-9- | Delta-9- | THCA-B | Yes |
| tetrahydrocannabinols | tetrahydrocannabinolic acid B | | |
| Delta-9- | Delta-9- | THCA-C4 | Yes |
| tetrahydrocannabinols | tetrahydrocannabinolic acid-C4 | | |
| Delta-9- | Delta-9- | THC-C1 | No |
| tetrahydrocannabinols | tetrahydrocannabiorcol | | |
| Delta-9- | Delta-9- | THCA-C1 | Yes |
| tetrahydrocannabinols | tetrahydrocannabiorcolic acid | | |
| Delta-9- | Delta-9- | THCV | No |
| tetrahydrocannabinols | tetrahydrocannabivarin | | |
| Delta-9- | Delta-9- | THCVA | Yes |
| tetrahydrocannabinols | tetrahydrocannabivarinic acid | | |
| Miscellaneous | 10-Oxo-delta-6a- | OTHC | No |
| cannabinoids | tetrahydrocannabinol | | |
| Miscellaneous | Cannabichromanon | CBCF | No |
| cannabinoids | | | |
| Miscellaneous | Cannabifuran | CBF | No |
| cannabinoids | | | |
| Miscellaneous | Cannabiglendol | | No |
| cannabinoids | | | |
| Miscellaneous | Cannabiripsol | CBR | No |
| cannabinoids | | | |
| Miscellaneous | Cannbicitran | CBT | No |
| cannabinoids | | | |
| Miscellaneous | Dehydrocannabifuran | DCBF | No |
| cannabinoids | | | |
| Miscellaneous | Delta-9-cis- | cis-THC | No |
| cannabinoids | tetrahydrocannabinol | | |
| Miscellaneous | Tryhydroxy-delta-9- | triOH-THC | No |
| cannabinoids | tetrahydrocannabinol | | |

TABLE 3
Cannabinoids with Carboxylic Acids and Corresponding Decarboxylated Products

| | | | |
|---|---|---|---|
| Cannabigerolic acid | CBGA | Cannabigerol | CBG |
| Δ9-tetrahydrocannabinolic acid | THCA | Δ9-tetrahydrocannabinol | THC |
| Cannabidiolic acid | CBDA | Cannabidiol | CBD |
| Cannabichromenenic acid | CBCA | Cannabichromene | CBC |
| Cannabigerovarinic acid | CBGVA | Cannabigerivarin | CBGV |
| Tetrahydrocanabivarinic acid | THCVA | Tetrahydrocannabivarin | THCV |
| Cannabidivarinic acid | CBDVA | Cannabidivarin | CBDV |
| Cannabichromevarinic acid | CBCVA | Cannabichromevarin | CBCV |

### II. Plant-Derived Pharmaceuticals

The present invention is also directed to plant-derived pharmaceuticals formed via the herein-described contaminant removal methods, wherein the resulting plant-derived pharmaceuticals (e.g., extracts) have a desired color (e.g., without extract darkening to a deep red color). The plant-derived pharmaceuticals include, but are not limited to, the cannabinoids listed in Tables 2 and 3 above.

### III. Methods of Using Plant-Derived Pharmaceuticals

The present invention is also directed towards methods of using plant-derived pharmaceuticals formed via the herein-described contaminant removal methods. Methods of using the plant-derived pharmaceuticals formed via the herein-described contaminant removal methods include, but are not limited to, methods of treating a patient with a disease or disorder, wherein the treatment comprises administering an effective amount of one or more of the plant-derived pharmaceuticals to the patient.

In one exemplary embodiment, the method of treated a patient with a disease or disorder comprises administering an effective amount of one or more pesticide-free cannabinoids, such as one or more of the cannabinoids shown in Tables 2 and 3, to the patient. A number of diseases and/or disorders may be treated using Cannabinoids including, but not limited to, (1) anxiety (i.e., to reduce anxiety), (2) inflammation (i.e., to reduce inflammation), (3) pain (i.e., to relief pain), (4) nausea and vomiting caused by chemotherapy (i.e., to reduce nausea and vomiting), (5) cancer (i.e., to kill cancer cells and slow tumor growth), (6) multiple sclerosis (i.e., to relax tight muscles in people with multiple sclerosis), and (7) appetite and weight gain (i.e., to stimulate appetite and improve weight gain in people with cancer and AIDS).

The present invention will be further described in the following additional embodiments, examples, and claims.

### ADDITIONAL EMBODIMENTS

### Methods of Removing Contaminants From Plant-Derived Pharmaceuticals

1. A method of removing contaminants from a plant-derived pharmaceutical, said method. comprising: reacting one or more contaminants within a plant-derived extract with a hydroxide in polar solvent to form one or more contaminant by-products in a reaction mixture; and separating the one or more contaminant by-products from the plant-derived extract so as to obtain a contaminant-reduced, plant-derived extract, wherein the hydroxide comprises ammonium hydroxide, a tetraalkylammonium hydroxide, or a combination thereof.
2. A method of removing contaminants from a plant-derived pharmaceutical, said method comprising: reacting one or more contaminants within a plant-derived extract with ammonium hydroxide in polar solvent to form one or more contaminant by-products in a reaction mixture; and separating the one or more contaminant by-products from the plant-derived extract so as to obtain a contaminant-reduced, plant-derived extract.
3. A method of removing contaminants from a plant-derived pharmaceutical derived from unfiltered alcoholic Cannabis extracts or distillates from California USA, said method comprising: reacting one or more contaminants within a plant-derived extract with a hydroxide in polar solvent to form one or more contaminant by-products in a reaction mixture; and separating the one or more contaminant by-products from the plant-derived extract so as to obtain a contaminant-reduced, plant-derived extract, wherein the hydroxide comprises ammonium hydroxide, a tetraalkylammonium hydroxide, or a combination thereof, and the contaminant-reduced, plant-derived extract does not have a red color.
4. The method of embodiment 1 or 3. wherein the hydroxide comprises ammonium hydroxide.
5. The method of embodiment 1 or 3, wherein the hydroxide comprises a tetraalkylammonium hydroxide.
6. The method of embodiment 5, wherein the tetraalkylammonium hydroxide comprises tetramethylammonium hydroxide.
7. The method of embodiment 5, wherein the tetraalkylammonium hydroxide comprises tetraethylammonium hydroxide.
8, The method of embodiment 5, wherein the tetraalkylammonium hydroxide comprises tetrapropylammonium hydroxide.
9. The method of any one of embodiments 1 to 8, wherein the one or more contaminants comprise one or more pesticides, one or more herbicides, one or more fungicides, or any combination thereof.
10. The method of any one of embodiments 1 to 9, wherein the one or more contaminants comprise one or more hydrolyzable pesticides.
11. The method of any one of embodiments 1 to 10, wherein the polar solvent comprises methanol, ethanol, n-propanol, isopropanol, butanol, dimethyl sulfoxide, dimethylformamide, or any combination thereof.
12. The method of any one of embodiments 1 to 11, wherein the polar solvent comprises methanol.
13. The method of any one of embodiments 1 to 12, further comprising: forming a polar solvent solution comprising the hydroxide or ammonium hydroxide in the polar solvent, wherein the hydroxide or ammonium hydroxide is present in the polar solvent at a concentration of from about 0:125 grams (g,) to 0.5 g of hydroxide or ammonium hydroxide per milliliter (ml) of polar solvent.
14. The method of any one of embodiments 1 to 13, wherein said reacting step comprises: adding the plant-derived extract to the hydroxide or ammonium hydroxide in the polar solvent to form the reaction mixture; and agitating the reaction mixture with mixing while (i) heating the reaction mixture to boiling for a period of time up to about one hour, (ii) ultrasonically vibrating the reaction mixture for a period of time up to about one hour, or (iii) both (i) and (ii).
15. The method of embodiment 14, wherein said method further comprises, following said agitating step: allowing the reaction mixture to cool down to below about 40°C.
16. The method of any one of embodiments 1 to 15. wherein said method does not comprise a neutralizing step acid after said reacting step (e.g., after the cooling step described in embodiment 15).
17. The method of any one of embodiments 1 to 16, wherein said separating step comprises: evaporating the polar solvent from the reaction mixture under reduced pressure so as to result in a polar solvent-free reaction mixture comprising (i) crystalline alkali metal acetate, and the plant-derived extract.
18. The method of embodiment 17, wherein said separating step further comprises: adding a first alkane to the polar solvent-free reaction mixture so as to form a non-aqueous liquid phase.
19. The method of embodiment 18, wherein from about 30 ml to about 120 ml of water is added to the polar solvent-free reaction mixture.
20. The method of embodiment 18 or 19, wherein about 40 ml of water is added to the polar solvent-free reaction mixture,
21. The method of any one of embodiments 18 to 20, wherein from about 30 ml to about 120 ml of the first alkane is added to the polar solvent-free reaction mixture.
22. The method of any one of embodiments 18 to 21, wherein about 40 ml of the first alkane is added to the polar solvent-free reaction mixture.
23. The method of any one of embodiments 18 to 22, wherein the first alkane is butane, pentane, cyclopentane, hexane, cyclohexane, heptane, octane, isooctane, or any combination thereof
24. The method of any one of embodiments 18 to 23, wherein the first alkane is heptane.
25. The method of any one of embodiments 18 to 24, further comprising: agitating the aqueous liquid phase and the non-aqueous liquid phase so as to dissolve (1) the crystalline alkali metal acetate in the aqueous liquid phase, and (2) the plant-derived extract in the non-aqueous liquid phase.

26. The method of any one of embodiments 18 to 25, further comprising: introducing the aqueous liquid phase and the non-aqueous liquid phase into a separatory funnel so as to form an upper non-aqueous liquid phase and a lower aqueous liquid phase; and separating the lower aqueous liquid phase from the upper non-aqueous liquid phase.

27. The method of embodiment 26, further comprising: passing the upper non-aqueous liquid phase through one or more columns with each column comprising one or more adsorbents; and collecting an effluent exiting the one or more columns.
28. The method of embodiment 27, wherein the one or more adsorbents comprise magnesium silicate (e.g., FLORISIL® adsorbent), activated alumina, magnesium salt of activated alumina, silica, bonded phase silica, polymeric non-ionic reversed phase resins, or any combination thereof.
29. The method of embodiment 27 or 28, wherein the one or more adsorbents comprise silica gel in a first column.
30. The method of any one of embodiments 27 to 29, wherein the one or more adsorbents comprise magnesium silicate (e.g., FLORISIL® adsorbent) in a second column.
31. The method of any one of embodiments 26 to 30, wherein each of the one or more columns comprises from about 25 g to about 60 g of the one or more adsorbents.
32. The method of any one of embodiments 26 to 31, wherein each of the one or more columns comprises about 40 g of the one or more adsorbents.
33. The method of any one of embodiments 26 to 32, further comprising: passing one or more column volumes of a rinse mixture through the one or more columns (e.g., an initial weak solvent (e.g., one or more second alkanes such as heptane) with or without some small percentage (e.g., 1-20wt %) of a stronger solvent (e.g., methanol, ethanol, isopropanol, n-propanol, ethyl acetate, acetone, dichloromethane, chloroform, toluene, and the like) to reduce collection volume but without eluting any residual undesirable non-Cannabinoid substances); and collecting the effluent exiting the one or more columns.
34. The method of embodiment 33, wherein the one or more column volumes comprises from about 40 ml to about 180 ml of the rinse mixture.
35. The method of embodiment 33 or 34, wherein the one or more column volumes comprises about 120 ml of the rinse mixture.
36. The method of any one of embodiments 33 to 35, wherein the second alkane and the ethyl acetate are present, by volume, at a ratio of 80:20 v/v.
37. The method of any one of embodiments 33 to 36, wherein the second alkane comprises heptane.
38. The method of any one of embodiments 27 to 37, further comprising: evaporating the effluent under reduced pressure so as to (1) remove one or more of: (i) the first alkane, (ii) the weak solvent (e.g., second alkane), and (iii) the stronger solvent (e.g., ethyl acetate), and (2) result in the contaminant-reduced plant-derived extract.
39. The method of any one of embodiments 1 to 38, further comprising: testing the contaminant-reduced plant-derived extract for possible detectable levels of one or more contaminants.
40. The method of any one of embodiments 1 to 39, wherein from about 5 g to about 25 _{g} of the plant-derived extract is reacted with the hydroxide or ammonium hydroxide in the alcohol.
41. The method of any one of embodiments 1 to 40, wherein about 10 g of the plant-derived extract is reacted with the hydroxide or ammonium hydroxide in the alcohol.
42. The method of any one of embodiments 1 to 2 and 4 to 41, wherein the plant-derived extract is a cannabis-derived extract.
43. The method of any one of embodiments 1 to 42, wherein the contaminant-reduced plant-derived extract comprises one or more cannabinoids.
44. The method of any one of embodiments 1 to 43, wherein the contaminant-reduced plant-derived extract does not have a red color.
45. The method of any one of embodiments 1 to 44, wherein the contaminant-reduced plant-derived extract has a yellowish anther “honey gold” color.
46. A method of removing contaminants from a cannabis plant-derived pharmaceutical, said method comprising: reacting one or more contaminants within a cannabis plant-derived extract with ammonium hydroxide in a polar solvent to form one or more contaminant by-products in a reaction mixture, wherein said reacting step comprises: adding the cannabis plant-derived extract to the ammonium hydroxide in the polar solvent to form the reaction mixture; agitating the reaction mixture with mixing; and separating the one or more contaminant by-products from the cannabis plant-derived extract so as to obtain a contaminant-reduced cannabis plant-derived extract, wherein said separating step comprises: adding a first alkane to the reaction mixture so as to form a non-aqueous liquid phase within the reaction mixture, wherein the first alkane comprises butane, pentane, cyclopentane, hexane, cyclohexane, heptane, octane, isooctane, or any combination thereof; and separating an upper non-aqueous liquid phase from a lower aqueous liquid phase, the upper non-aqueous liquid phase comprising the first alkane and the contaminant-reduced cannabis plant-derived extract, wherein the contaminant-reduced cannabis plant-derived extract comprises one or more cannabinoids.
47. The method of embodiment 46, wherein the one or more contaminants comprise one or more pesticides, one or more herbicides, one or more fungicides, or any combination thereof.
48. The method of embodiment 46 or 47, wherein the one or more contaminants comprise one or more hydrolyzable pesticides.
49. The method of any one of embodiments 46 to 48, wherein the polar solvent comprises methanol; and the first alkane comprises hexane, heptane, or any combination thereof.
50. The method of any one of embodiments 46 to 49, further comprising: passing the upper non-aqueous liquid phase through one or more columns with each column comprising one or more adsorbents; and collecting an effluent exiting the one or more columns.
51. The method of embodiment 50, wherein the one or more adsorbents comprise magnesium silicate, activated alumina, magnesium salt of activated alumina, sodium sulfate, silica, bonded phase silica, polymeric non-ionic reversed phase resins, or any combination thereof.
52 The method of embodiment 51, wherein the one or more adsorbents comprise (i) silica gel in a first column, and (ii) magnesium silicate in a second column.
53. The method of embodiment 50, further comprising: passing one or more column volumes of a rinse mixture through the one or more columns, the rinse mixture comprising heptane and ethyl acetate at a heptane:ethyl acetate ratio of about 80:20 by volume; and collecting the rinse mixture exiting the one or more columns.
54. The method of embodiment 53, further comprising: evaporating the rinse mixture under reduced pressure so as to (1) remove one or more of: (i) the first alkane, heptane, and (iii) ethyl acetate, and (2) result in the contaminant-reduced cannabis plant-derived extract.
55. A method of removing contaminants from a plant-derived pharmaceutical, said method comprising: reacting one or more contaminants within a plant-derived extract with ammonium hydroxide in a polar solvent to form one or more contaminant by-products in a reaction mixture; and separating the one or more contaminant by-products from the reaction mixture so as to obtain a contaminant-reduced plant-derived extract, wherein said reacting step comprises: adding the plant-derived extract to the ammonium hydroxide in the polar solvent to form the reaction mixture; and agitating the reaction mixture with mixing; and wherein said separating step comprises: adding a first alkane to the reaction mixture so as to form a non-aqueous liquid phase within the reaction mixture; separating an aqueous liquid phase of the reaction mixture from the non-aqueous liquid phase; passing the non-aqueous liquid phase through one or more columns with each column comprising one or more adsorbents; passing one or more column volumes of a rinse mixture through the one or more columns, the rinse mixture comprising a second alkane and ethyl acetate; and evaporating the rinse mixture under reduced pressure so as to (1) remove one or more of: (i) the first alkane, (ii) the second alkane, and (iii) ethyl acetate, and (2) result in the contaminant-reduced plant-derived extract.
56. The method of embodiment 55, wherein (a) the second alkane comprises heptane, and (h) the rinse mixture comprises (i) the heptane, and (ii) up to about 20 weight percent (wt %) of the ethyl acetate.
57. The method of embodiment 55 or 56, wherein the one or more contaminants comprise one or more pesticides, one or more herbicides, one or more fungicides, or any combination thereof.
58. A method of removing contaminants from a plant-derived pharmaceutical, said method comprising: reacting one or more contaminants within a plant-derived extract with ammonium hydroxide in a polar solvent to form one or more contaminant by-products in a reaction mixture, the one or more contaminants comprising one or more pesticides, one or more herbicides, one or more fungicides, or any combination thereof; and separating (i) the one or more contaminant by-products, and (ii) a contaminant-reduced plant-derived extract from the reaction mixture, wherein said separating step comprises: adding a first alkane to the reaction mixture so as to form a non-aqueous liquid phase within the reaction mixture; separating an aqueous liquid phase of the reaction mixture from the non-aqueous liquid phase; passing the non-aqueous liquid phase through one or more columns with each column comprising one or more adsorbents; passing one or more column volumes of a rinse mixture through the one or more columns, the rinse mixture comprising a second alkane and ethyl acetate; and evaporating the rinse mixture under reduced pressure so as to (1) remove one or more of: (i) the first alkane, (ii) the second alkane, and (iii) ethyl acetate, and (2) result in the contaminant-reduced plant-derived extract.
59. The method of embodiment 58, wherein said reacting step comprises: adding the plant-derived extract to the ammonium hydroxide in the polar solvent to form the reaction mixture; and agitating the reaction mixture with mixing.
60. The method of any one of embodiments 46 to 59, wherein the one or more contaminants comprise one or more hydrolyzable pesticides.
61. The method of any one of embodiments 46 to 60, wherein the contaminant-reduced plant-derived extract comprises one or more cannabinoids.

### Contaminant-Reduced Plant-Derived Pharmaceuticals

62. A contaminant-reduced plant-derived extract formed by the method of any one of embodiments 1 to 61.

### Methods of Using Contaminant-Reduced Plant-Derived Pharmaceuticals

63. A method of using the contaminant-reduced plant-derived extract formed by the method of any one of embodiments 1 to 61, said method comprising: treating a patient suffering from a disease or disorder by administering an effective amount of the contaminant-reduced plant-derived extract to the patient.
64. The method of embodiment 63, wherein the effective amount of the contaminant-reduced plant-derived extract is orally provided to the patient.
65. The method of embodiment 63 or 64, wherein the effective amount of the contaminant-reduced plant-derived extract is intravenously provided to the patient.
66. The method of any one of embodiments 63 to 65, wherein the disease or disorders comprises (1) anxiety (i.e., to reduce anxiety), (2) inflammation (i.e., to reduce inflammation), (3) pain (i.e., to relief pain), (4) nausea and vomiting caused by chemotherapy (i.e., to reduce nausea and vomiting), (5) cancer (i.e., to kill cancer cells and slow tumor growth), (6) multiple sclerosis (i.e., to relax tight muscles in people with multiple sclerosis), (7) appetite and weight gain (i.e., to stimulate appetite and improve weight gain in people with cancer and AIDS), or any combination thereof.

The present invention is further illustrated by the following example, which is not to be construed in any way as imposing limitations upon the scope thereof On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

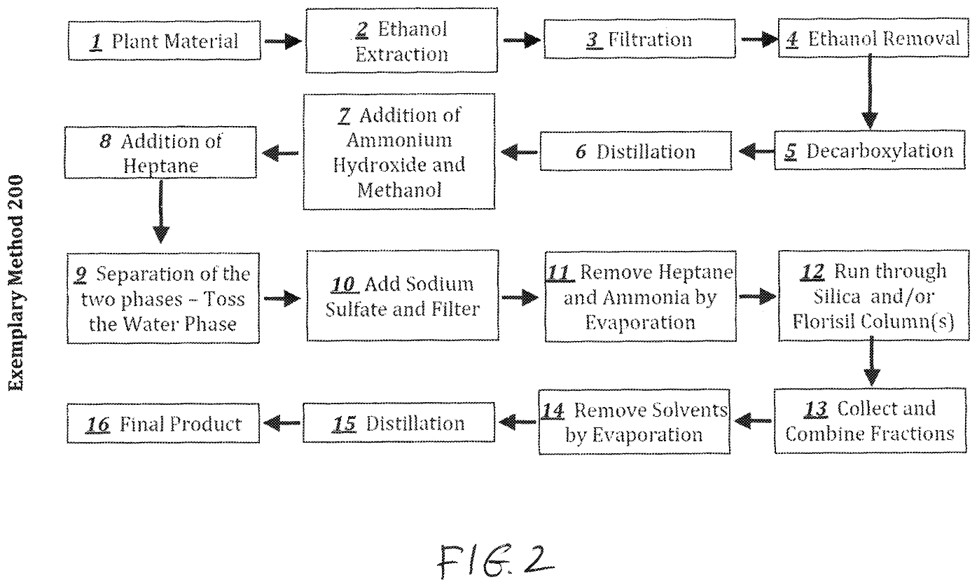

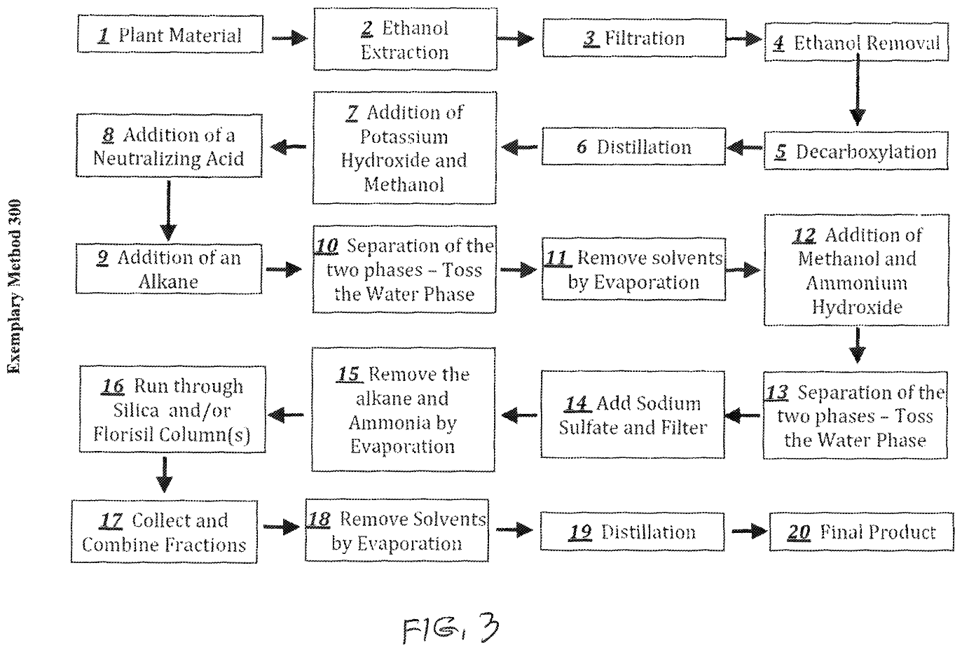

Methods of removing contaminants from cannabis-derived extracts were conducted using the exemplary method 200 shown in FIG. 2. The method steps were conducted as follows:

1) add 50 g of cannabis extract to 500 mL of methanol and dissolve;

2) add 200 mL, of 28% ammonium hydroxide to the mixture and agitate for 30 to 120 min;

3) add 250 mL of water;

4) add 175 mL of heptane and agitate—an emulsion may appear which will disappear in 10 to 45 min;

5) use a separatory funnel to recover the upper heptane layer;

6) add 175 mL of heptane and agitate—an emulsion may appear which will disappear in 10 to 45 min;

7) use a separatory funnel to recover the upper heptane layer;

8) combine the two recovered heptane layers;

9) add 25 to 100 g of sodium sulfate to the two recovered heptane layers;

10) agitate the mixture and filter off the sodium sulfate;

11) evaporate the heptane under reduced pressure (Note that while there is some ammonia gas in the recovered heptane layers, the ammonia gas will be removed during this step.);

12) redissolve the extract in 50 mL of heptane and load the entirety of the extract to a 330 g chromatographic column packed with 330 g of normal phase silica gel followed by a 330 g chromatographic column of FLORISIL® (i.e., magnesium silicate) both of which are preconditioned with heptane;

13) elute the cannabinoids from the two-column set with 3 column volumes of a mixture of 85% heptane with 15% ethyl acetate;

14) remove the eluting solvents under reduced pressure; and

15) distill the remaining extract to (i) remove residual solvents, and (ii) obtain a yellowish “honey gold” product that is contaminant free.

The following observations were made during the process steps, referring to the numbering shown in FIG. 2:

At step 7, the solution first had a purplish color. Then, it turned a brownish color.

At step 9, the water phase was brownish colored.

All ammonia gas in the heptane at step 9 was removed by the evaporation under reduced pressure.

The extract at step 11 was slightly brownish colored. Note that if the extract s dark brown at step 11, a wash with 1N HCl removes most, if not all, of the brown color and turns the extract to a yellowish amber.

The extract at step 14 had a yellowish amber.

TLC and RP-HPLC analysis of the extract at steps 11, 14, and 16 revealed that all the Cannabinoids were present.

When 100 g of extract was treated with 80-85% Cannabinoid content, about 90 grams were recovered at step 16 with 90-95% Cannabinoid content.

The extract at step 16 was a desired yellowish amber “honey gold” product.

The following quantities were used in this procedure:

| Initial Reactant | | |
|---|---|---|
| Extract | FW | 314.00 |
| Solvent | | |
| Methanol to Extract Ratio | ml/g | 5.00 |
| Hydroxide Ions | | |
| Ratio of Ammonium Hydroxide Volume | % | 100.0% |
| to Extract Weight by Percent | | |
| Ammonium Hydroxide | FW | 35.05 |
| Solvents | | |
| Water | % | 100% |
| Heptane Ratio | % | 25% |
| Initial Reactant | | |
| Extract | g | 50 |
| Extract | mm | 159 |
| Pesticides in Extract | ppm | 25 |
| Pesticides in Extract | g | 1.25E−03 |
| Pesticides in Extract | MW | 300 |
| Pesticides in Extract | mM | 4.17E−03 |
| Solvent | | |
| Methanol Volume | mL | 250 |
| Hydroxide Ions | | |
| Ammonium Hydroxide | g | 50 |
| Ammonium Hydroxide | mM | 1,427 |
| Ammonium Hydroxide | wt % | 28% |
| Ammonium Hydroxide | g | 179 |
| Ammonium Hydroxide Density | g/mL | 0.90 |
| Ammonium Hydroxide Volume | mL | 193 |
| Ratio Water/(Methanol + Water) | % | 44% |
| Water Added | mL | 250 |
| Total Volume | mL | 698 |
| Solvent | | |
| Heptane Extraction 1 | mL | 175 |
| Heptane Extraction 2 | mL | 175 |
| Total Volume of Heptane | mL | 349 |
| Molar Excess of Hydroxide Ions to Pesticides | | |
| Ammonium Hydroxide | # | 342,368 |

### EXAMPLE 3

Methods of removing contaminants from cannabis-derived extracts were conducted using the exemplary method 300 shown in FIG. 3. Exemplary method 300 comprises method steps from (1) the method disclosed in U.S. Pat. No. 11,026,985, the subject matter of which is hereby incorporated by reference in its entirety into the present patent application (e.g., steps 1-11 of exemplary method 300 shown in FIGS. 3), and (2) the herein-described method (e.g., steps 12-20 of exemplary method 300 shown in FIG. 3).

In addition, it should be understood that although the above-described methods of removing contaminants from plant-derived pharmaceuticals, plant-derived pharmaceuticals and methods of using plant-derived pharmaceuticals are described as “comprising” one or more components or steps, the above-described methods of removing contaminants from plant-derived pharmaceuticals, plant-derived pharmaceuticals and methods of using plant-derived pharmaceuticals may “comprise,” “consists of,” or “consist essentially of” the above-described method steps and components of the methods of removing contaminants from plant-derived pharmaceuticals, plant-derived pharmaceuticals and methods of using plant-derived pharmaceuticals. Consequently, where the present invention, or a portion thereof, has been described with an open-ended term such as “comprising,” it should be readily understood that (unless otherwise stated) the description of the present invention, or the portion thereof, should also be interpreted to describe the present invention, or a portion thereof, using the terms “consisting essentially of” or “consisting of” or variations thereof as discussed below.

As used herein, the terms “comprises,” “comprising,” “includes,” “including,” “has,” “having,” “contains,” “containing,” “characterized by” or any other variation thereof, are intended to encompass a non-exclusive inclusion, subject to any limitation explicitly indicated otherwise, of the recited components. For example, a method of removing contaminants from plant-derived pharmaceuticals, plant-derived pharmaceutical, and/or method of using plant-derived pharmaceuticals that “comprises” a list of elements (e.g., components or steps) is not necessarily limited to only those elements (or components or steps), but may include other elements (or components or steps) not expressly listed or inherent to the method of removing contaminants from plant-derived pharmaceuticals, plant-derived pharmaceutical, and/or method of using plant-derived pharmaceuticals.

As used herein, the transitional phrases “consists of” and consisting of exclude any element, step, or component not specified. For example, “consists of” or “consisting of” used in a claim would limit the claim to the components, materials or method steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a method of removing contaminants from plant-derived pharmaceuticals, plant-derived pharmaceutical, and/or method of using plant-derived pharmaceuticals that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of.”

Further, it should be understood that the herein-described methods of removing contaminants from plant-derived pharmaceuticals, plant-derived pharmaceuticals, and/or methods of using plant-derived pharmaceuticals may comprise, consist essentially of, or consist of any of the herein-described components, method steps, and/or features, as shown in the figures with or without any feature(s) not shown in the figures. In other words, in some embodiments, the methods of removing contaminants from plant-derived pharmaceuticals, plant-derived pharmaceuticals, and/or methods of using plant-derived pharmaceuticals of the present invention do not have any additional features other than those shown in the figures, and such additional features, not shown in the figures, are specifically excluded from the methods of removing contaminants from plant-derived pharmaceuticals, plant-derived pharmaceuticals, and/or methods of using plant-derived pharmaceuticals. In other embodiments, the methods of removing contaminants from plant-derived pharmaceuticals, plant-derived pharmaceuticals, and/or methods of using plant-derived pharmaceuticals of the present invention do have one or more additional features that are not shown in the figures.

The present invention is described above and further illustrated below by way of claims, which are not to be construed in any way as imposing limitations upon the scope of the invention. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

What is claimed is:

1. A method of removing contaminants from a cannabis plant-derived pharmaceutical, said method comprising:
reacting one or more contaminants within a cannabis plant-derived extract with a hydroxide in polar solvent to form one or more contaminant by-products in a reaction mixture; and
separating the one or more contaminant by-products from the cannabis plant-derived extract so as to obtain a contaminant-reduced, cannabis plant-derived extract,
wherein the hydroxide comprises ammonium hydroxide, a tetraalkylammonium hydroxide, or a combination thereof, and
wherein the contaminant-reduced cannabis plant-derived extract comprises one or more cannabinoids.

2. The method of claim 1, wherein the hydroxide comprises ammonium hydroxide or a tetraalkylammonium hydroxide.

3. The method of claim 1, wherein the polar solvent comprises methanol, ethanol, n-propanol, isopropanol, butanol, dimethyl sulfoxide, dimethylformamide, or any combination thereof.

4. The method of claim 1, further comprising:
forming a polar solvent solution comprising the hydroxide in the polar solvent,
wherein the hydroxide is present in the polar solvent at a concentration of from about 0.125 grams (g) to 0.5 g of hydroxide per milliliter (nil) of polar solvent.

5. The method of claim 1, wherein said reacting step comprises:
adding the plant-derived extract to the hydroxide in the polar solvent to form the reaction mixture; and
agitating the reaction mixture with mixing while (i) heating the reaction mixture to boiling for a period of time up to about one hour, (ii) ultrasonically vibrating the reaction mixture for a period of time up to about one hour, or (iii) both (i) and (ii).

6. The method of claim 1, wherein said method does not comprise a neutralizing step acid after said reacting step.

7. The method of claim 1, wherein said separating step comprises:
evaporating the polar solvent from the reaction mixture under reduced pressure so as to result in a polar solvent-free reaction mixture comprising (i) crystalline alkali metal acetate, and (ii) the plant-derived extract.

8. The method of claim 7, wherein said separating step further comprises:
adding a first alkane to the polar solvent-free reaction mixture so as to form a non-aqueous liquid phase,
wherein the first alkane is butane, pentane, cyclopentane, hexane, cyclohexane, heptane, octane, isooctane, or any combination thereof.

9. The method of claim 8, further comprising:
introducing the aqueous liquid phase and the non-aqueous liquid phase into a separatory funnel so as to form an upper non-aqueous liquid phase and a lower aqueous liquid phase; and
separating the lower aqueous liquid phase from the upper non-aqueous liquid phase,

10. The method of claim 9, further comprising:
passing the upper non-aqueous liquid phase through one or more columns with each column comprising one or more adsorbents; and
collecting an effluent exiting the one or more columns.

11. The method of claim 10, further comprising:
passing one or more column volumes of a rinse mixture through the one or more columns (e.g., an initial weak solvent (e.g., one or more second alkanes such as heptane) with or without some small percentage (e.g., 1-20 wt %) of a stronger solvent (e.g., methanol, ethanol, isopropanol, n-propanol, ethyl acetate, acetone, dichloromethane, chloroform, toluene, and the like) to reduce collection volume but without eluting any residual undesirable non-Cannabinoid substances); and
collecting the effluent exiting the one or more columns.

12. The method of claim 11, further comprising:
evaporating the effluent under reduced pressure so as to (1) remove one or more of: (i) the first alkane, (ii) the weak solvent (e.g., second alkane), and (iii) the stronger solvent (e.g., ethyl acetate), and (2) result in the contaminant-reduced plant-derived extract.

13. The method of claim 1, further comprising:
testing the contaminant-reduced plant-derived extract for possible detectable levels of one or more contaminants.

14. The method of claim 1, wherein about 1©g of the plant-derived extract is reacted with the hydroxide in the alcohol.

15. The method of claim 1, wherein the contaminant-reduced plant-derived extract does not have a red color, and the contaminant-reduced plant-derived extract has a yellowish amber “honey gold” color.

16. A method of removing contaminants from a cannabis plant-derived pharmaceutical, said method comprising:
reacting one or more contaminants within a cannabis plant-derived extract with ammonium hydroxide in polar solvent to form one or more contaminant by-products in a reaction mixture, the one or more contaminants comprise one or more pesticides, one or more herbicides, one or more fungicides, or any combination thereof; and
separating (i) the one or more contaminant by-products from (ii) the cannabis plant-derived extract so as to obtain a contaminant-reduced, cannabis plant-derived extract,
wherein the contaminant-reduced cannabis plant-derived extract comprises one or more cannabinoids.

17. A method of removing contaminants from a cannabis plant-derived pharmaceutical, said method comprising:
reacting one or more contaminants within a cannabis plant-derived extract with ammonium hydroxide in a polar solvent to form one or more contaminant by-products in a reaction mixture, the one or more contaminants comprising one or more pesticides, one or more herbicides, one or more fungicides, or any combination thereof, wherein said reacting step comprises:
adding the cannabis plant-derived extract to the ammonium hydroxide in the polar solvent to form the reaction mixture:
agitating the reaction mixture with mixing; and
separating the one or more contaminant by-products from the cannabis plant-derived extract so as to obtain a contaminant-reduced cannabis plant-derived extract, wherein said separating step comprises:
adding a first alkane to the reaction mixture so as to form a non-aqueous liquid phase within the reaction mixture, wherein the first alkane comprises butane, pentane, cyclopentane, hexane, cyclohexane, heptane, octane, isooctane, or any combination thereof; and
separating an upper non-aqueous liquid phase from a lower aqueous liquid phase, the upper non-aqueous liquid phase comprising the first alkane and the contaminant-reduced cannabis plant-derived extract, wherein the contaminant-reduced cannabis plant-derived extract comprises one or more cannabinoids.

18. The method of claim 17, further comprising:
passing the upper non-aqueous liquid phase through one or more columns with each column comprising one or more adsorbents; and
collecting an effluent exiting the one or more columns, wherein the one or more adsorbents comprise (i) silica gel in a first column, and (ii) magnesium silicate in a second column.

19. A contaminant-reduced plant-derived extract formed by the method claim 1.

20. A method of using the contaminant-reduced plant-derived extract formed by the method of claim 1, said method comprising:
treating a patient suffering from a disease or disorder by administering an effective amount of the contaminant-reduced plant-derived extract to the patient, wherein the disease or disorders comprises (1) anxiety (i.e., to reduce anxiety), (2) inflammation (i.e., to reduce inflammation), (3) pain (i.e., to relief pain), (4) nausea and vomiting caused by chemotherapy (i.e., to reduce nausea and vomiting), (5) cancer (i.e., to kill cancer cells and slow tumor growth), (6) multiple sclerosis (i.e., to relax tight muscles in people with multiple sclerosis), (7) appetite and weight gain (i.e., to stimulate appetite and improve weight gain in people with cancer and AIDS), or any combination thereof.
